(19) United States
(12) Patent Application Publication
(10) Pub. No.: US 2021/0368141 A1
(43) Pub. Date: Nov. 25, 2021

(54) SYSTEM AND METHOD FOR MULTI-SENSOR THREAT DETECTION PLATFORM

(71) Applicant: PatriotOne Technologies, Toronto (CA)

(72) Inventors: James Ashley STEWART, Saint John (CA); Shawn MITCHELL, Saint John (CA); Matthew Aaron Rogers CARLE, Fredericton (CA)

(21) Appl. No.: 17/329,822

(22) Filed: May 25, 2021

Related U.S. Application Data
(60) Provisional application No. 63/029,605, filed on May 25, 2020.

(51) Int. Cl. H04N 7/18; G08B 13/00; A61B 5/01; A61B 5/00

(52) U.S. Cl. CPC H04N 7/183; A61B 5/7275; A61B 5/01; G08B 13/00

(57) ABSTRACT

Embodiments described herein relate to a threat detection system and platform. This platform may use multi-sensors and radar technologies, in conjunction with an artificial intelligence system, to detect concealed and visible weapons such as guns and knives. The system may also detect health risk-based threats, through sensing of factors such as the absence of face masks, the presence of fever, or non-compliance with social distancing rules. Systems for violence detection, facilities support, tactical support and support of other industries are disclosed.

### CROSS REFERENCE TO RELATED APPLICATIONS

The application claims priority to and the benefit of U.S. Provisional Patent Application Ser. No. 63/029,605, entitled “SYSTEM AND METHOD FOR MULTI-SENSOR THREAT DETECTION PLATFORM”, filed on May 25, 2020, the disclosure of which is incorporated herein by reference in its entirety

### BACKGROUND

The embodiments described herein relate to security and surveillance, in particular, technologies related to video recognition threat detection.

Existing threat detection systems simply use motion or other triggers to focus cameras in front of a user, and in some cases places a highlight box around the subject of interest. Artificial intelligence (AI) technologies work best in support of humans, excelling where their human counterparts do not. AI excels at automating the mundane tasks, and tirelessly performing these monotonous, repetitive tasks.

A multi-sensor threat detection platform or system should allow for more effective resourcing, improved safety, crime reduction and asset protection. This platform should also be complemented by AI to free security teams from endless hours of monitoring tasks and allow them to engage in more effective and active security practices.

Such systems currently target specific risks, rather than holistic threat detection, and therefore cannot be easily leveraged to also detect health or other risks.

### SUMMARY

Embodiments described herein relate to a threat detection system and platform. This platform may use multiple-sensors and sensors of differing types including radar technologies, in conjunction with an artificial intelligence system, to detect concealed weapons such as guns and knives. The system may also detect health risk-based threats, through sensing of factors such as the absence of face masks, the presence of fever, atypical movement, or non-compliance with social distancing rules. Systems for violence detection, facilities support, tactical support and support of other industries are disclosed.

### BRIEF DESCRIPTION OF THE DRAWINGS

FIG. 1 is a diagram describing the requirements of a multi-sensor threat detection platform.

FIG. 2 is a diagram describing the importance of camera location.

FIG. 3 is a table describing camera capabilities.

FIG. 4 is a table describing high level roadmap and features.

FIG. 5 is a diagram illustrating a Phone Home Data Collection module.

FIG. 6 is a diagram illustrating dashboards of an exemplary system.

FIG. 7 is a diagram illustrating the Security Assist module.

FIG. 8 is a diagram illustrating the Tactical View module.

FIG. 9 is a diagram illustrating a private cloud concept.

FIG. 10 is a diagram illustrating a Mobile module.

FIG. 11 is a diagram illustrating modules for Health Risk Screening.

FIG. 12 is a diagram illustrating a workflow for Health Risk Screening.

FIG. 13 is a diagram illustrating a workflow for Mask Tracking.

FIG. 14 is a diagram illustrating deadlines for Pandemic Screening Timeline.

FIG. 15 is a diagram illustrating actions related to Elevated Body Temperature tasks.

FIG. 16 is a diagram illustrating actions related to Mask Detection tasks.

FIG. 17 is a diagram illustrating modules for Violence Detection.

FIG. 18 is a diagram illustrating a Fight Detection module.

FIG. 19 is a diagram illustrating a Disturbance Detection module.

FIG. 20 is a diagram illustrating modules for Facility Support.

FIG. 21 is a diagram illustrating modules to support additional verticals.

FIG. 22 is a system diagram of an exemplary threat detection system.

### DETAILED DESCRIPTION

In a preferred embodiment, a multi-sensor covert threat detection system is disclosed. This covert threat detection system utilizes software, artificial intelligence and integrated layers of diverse sensor technologies (e.g., cameras, etc.) to deter, detect and defend against active threats (e.g., detection of guns, knives or fights) before these threat events occur.

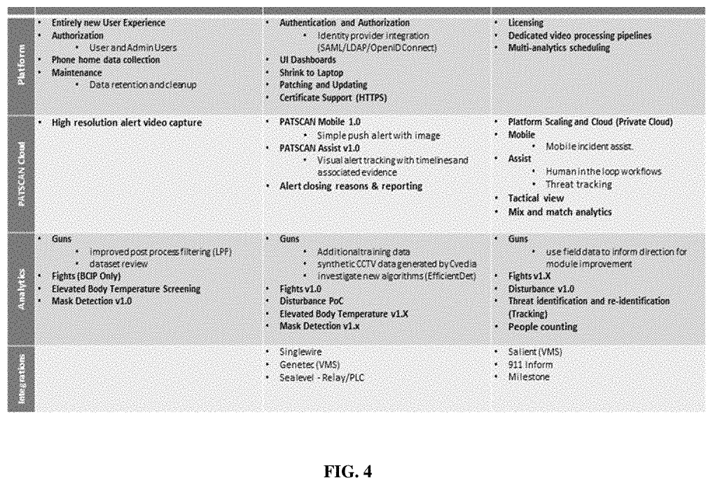

The threat detection system may allow the system operator to easily determine if the system is operational without requiring testing with actual triggering events. This system may also provide more situational information to the operator in real time as the incident is developing, showing them threat status and location, among other data, and show that information in a timely manner. A roadmap and feature set of an exemplary multi-sensor covert threat detection system is disclosed in FIG. 4.

### Multi-Sensor Threat Detection Platform Requirements:

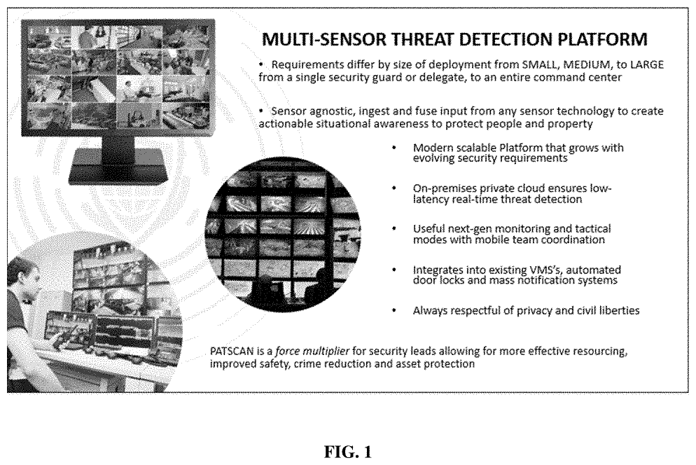

FIG. 1 is a diagram describing the capabilities of the multi-sensor threat detection platform. As seen in FIG. 1, the multi-sensor threat detection platform or system has the following capabilities, including:
Capabilities for different size deployments: from small, medium to large, and from a single security guard or delegate to an entire command center.
Sensor agnostic, able to ingest and combine input from multiple sensor technologies to create actionable situational awareness to protect people and property.
Modern scalable Platform that grows with evolving security requirements
On-premises private cloud ensures low-latency real-time threat detection and reduces connection vulnerability.
Useful next-gen monitoring and tactical modes with mobile team coordination
Integrates into existing Video Management System's automated door locks and mass notification systems
Respectful of privacy and civil liberties through anonymization of identifying information.

### Different Approach:

Distinguishing everyday objects and activities from true threats requires a lot more than a catalog of pictures. Many questions (e.g., Where is the object? Is it being carried? How is it being carried? How is the individual moving?) need to be answered in order to truly identify a threat in any given environment. The answer to all these questions is what provides context around what is being observed.

Context enables a multi-sensor threat platform to identify threats. Context enables the platform AI to generalize its understanding of threats and apply the AI to scenarios and environments it has never encountered in the past.

### Camera Location is Key to Success:

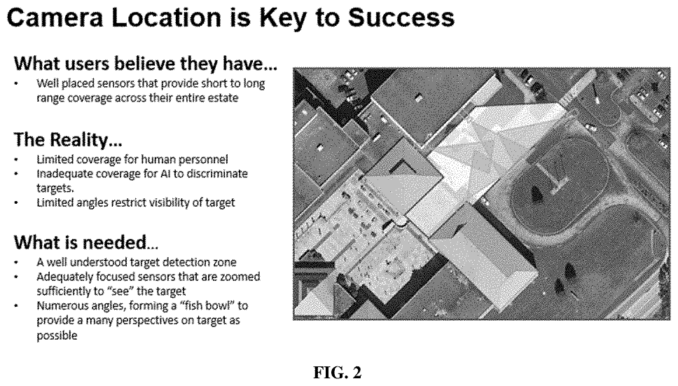

FIG. 2 is a diagram describing the importance of camera location. Users believe that they have well placed sensors that provide short to long range coverage across their entire estate. However, these sensors may have limited coverage for human personnel, Inadequate coverage for AI to discriminate targets, and limited angles restrict visibility of target.

What is needed is a system or platform, such as this platform, that has a well understood target detection zone, an adequate number of focused sensors that are zoomed and focused sufficiently to “see” the target, providing numerous angles, and forming a “fishbowl” to provide as many perspectives on target as possible.

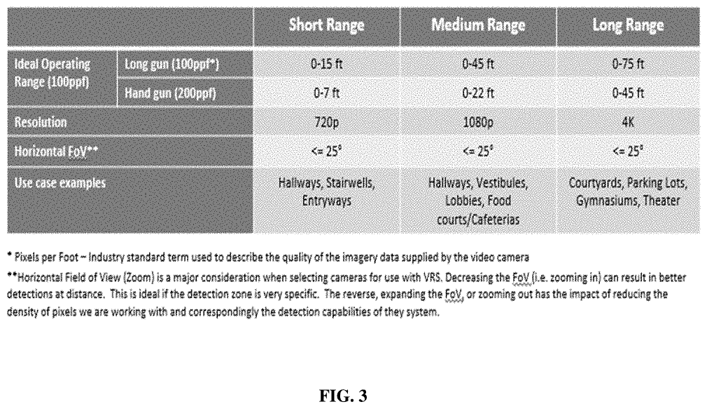

FIG. 3 is a table describing camera capabilities. It is crucial to match camera capabilities with the appropriate location in order to achieve optimal detection of threats in an environment. Current technological capabilities and costs, camera capabilities and suitability may be summarized in FIG. 3.

### Phone Home Data Collection:

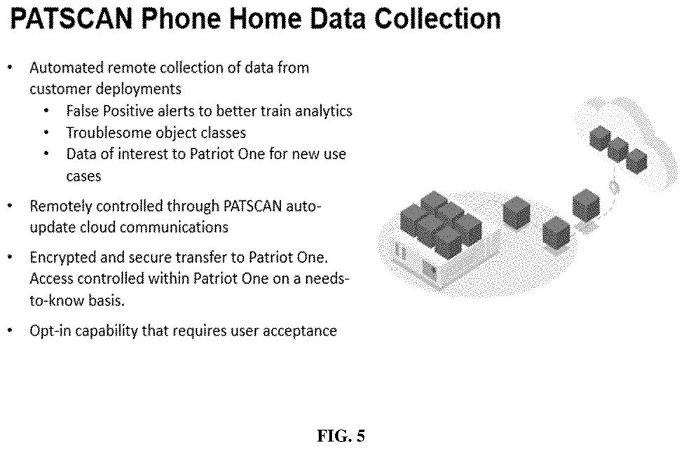

Embodiments of the multi-sensor threat detection platform may include features for a phone home data collection. FIG. 5 is a diagram illustrating a Phone Home Data Collection module, including the following features:
Automated remote collection of data from customer deployments
   False Positive alerts to better train analytics
   Troublesome object classes
   Data of interest for new use cases
Remote control through the platform's auto-update cloud communications or some other system
Encrypted and secure transfer to the service provider or some other central location or service. Access controlled within the service provider or within the service or location on a needs-to-know basis.
Opt-in capability that requires user acceptance

### Platform Dashboards:

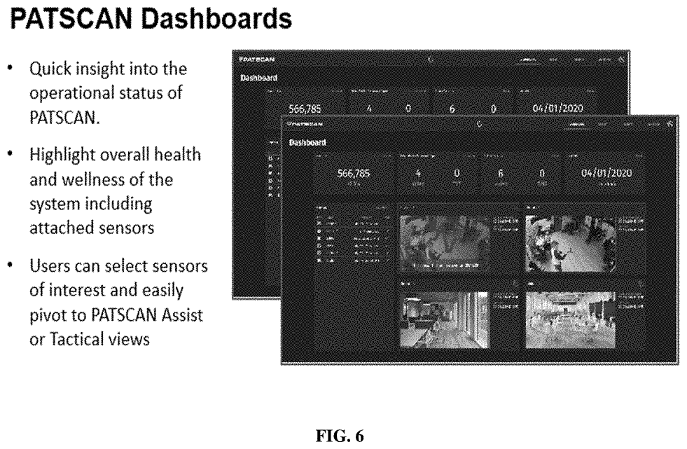

FIG. 6 is a diagram illustrating dashboards of an exemplary system. As seen in FIG. 6, the platform (or system) also includes dashboard screens that may provide any of the following:
Quick insight into the operational status of the platform.
Highlighting of overall health and wellness of the system including attached sensors
Ability for users to select sensors of interest and easily pivot to the platforms Assist or Tactical views

### Platform Security Assist:

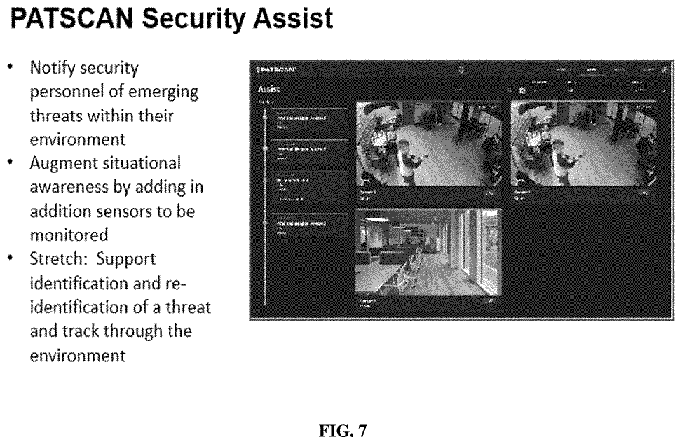

FIG. 7 is a diagram illustrating the Security Assist module. As seen in FIG. 7, the system also has Security Assist module that:
Notify security personnel of emerging threats within their environment
Augment situational awareness by adding in addition sensors to be monitored
Support identification and re-identification of a threat and track through the environment

### Platform Tactical View:

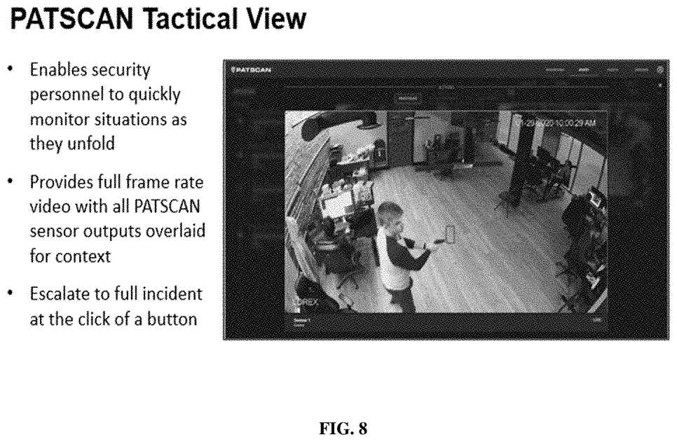

FIG. 8 is a diagram illustrating the Tactical View module. As seen in FIG. 8, the system also has Tactical View module that:
Enable security personnel to quickly monitor situations as they unfold
Provide full frame rate video with all sensor outputs overlaid for context
Escalate to full incident at the click of a button

### Private Cloud:

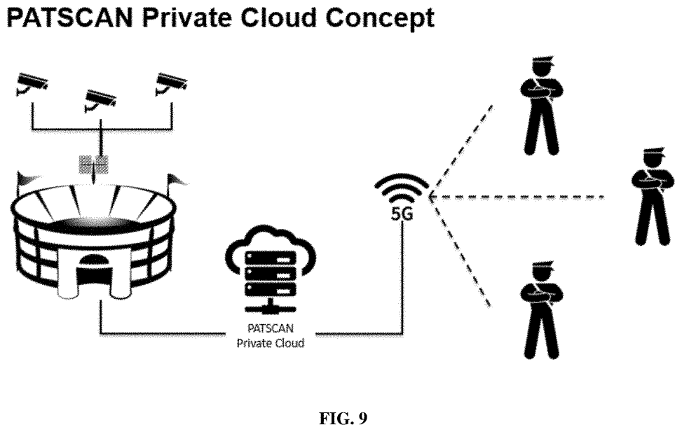

FIG. 9 is a diagram illustrating a private cloud concept. The system also has a private cloud offering that is:
Scalable, Private and Secure: On-premises private cloud of platform appliance to delivery threat detection at scale. All without the privacy concerns of public cloud infrastructures.
Self-Managed: No specialized skills are required to manage a cloud cluster. Simply plug in computing power as needed and the system will do the rest.
High availability: The cloud forms a redundant backend, ensuring that a hardware failure doesn't leave an organization blind to threats in their environment.
A sound investment: the cloud grows incrementally to meet customers' needs and changing environments.

### Mobile:

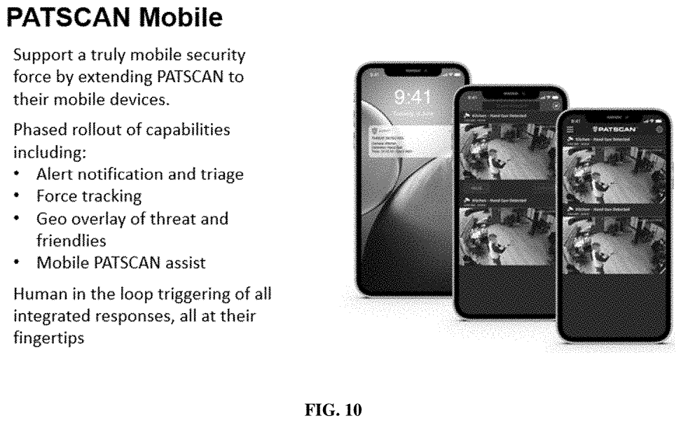

FIG. 10 is a diagram illustrating a Mobile module. The system also supports a mobile security force by extending at least some of its functionality to mobile applications on mobile devices. Users of the platform are kept in the loop by triggering of all the integrated responses, all available on mobile at their fingertips.

Further, the mobile version of the platform also has phased rollout of capabilities including:

Alert notification and triage

Force tracking

Geo overlay of threat and friendlies

Mobile assist

### Modules for Health Risk Screening:

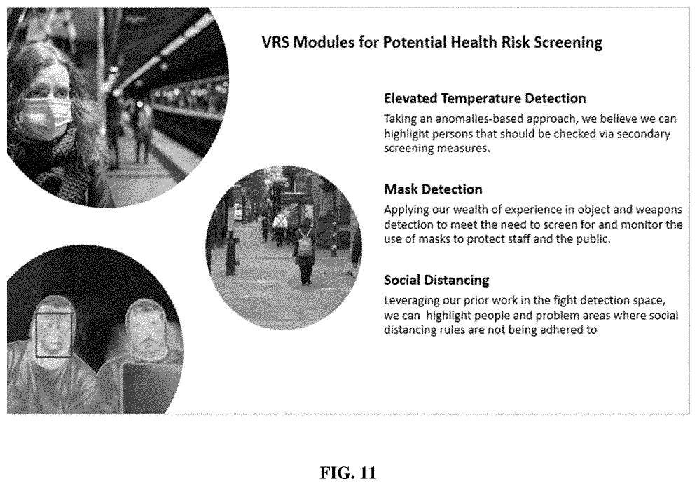

From “critical” organizations that remain open during a pandemic to most business that are opening their doors for the first time in months, social distancing is a reality and a new way of doing business. FIG. 11 is a diagram illustrating modules for Health Risk Screening. The system can provide assistance, support and analytics with health risk screening, by supporting the following modules:
Elevated Body Temperature Screening
   Using an anomalies-based approach, the system may highlight persons that should be checked via secondary screening measures.
   Screening AI for broader non-invasive temperature checks to protect locations and to facilitate the reopening of non-essential locations.
   Enable locations to implement new screening processes and capabilities to continue flattening the curve and reducing the risk of transmission of a pathogen.
Mask/No-Mask Tracking
   Ability to screen for and monitor the use of masks to protect staff and the public.
   Screening AI to support facilities enforce government requirements for utilization of non-medical masks in public areas.
   Assist with airline authorities' and larger commercial entities' efforts to make masks mandatory for customers, extending this capability to broad cross section of the corporate landscape
Social Distancing
   Ability to detect and highlight people and problem areas where social distancing rules are not being adhered to
   Screen AI to support facility teams to enforce social distancing recommendations to reduce virus spread

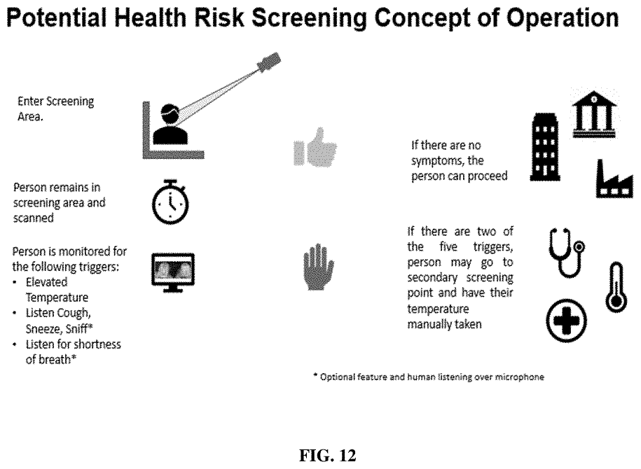

FIG. 12 is a diagram illustrating a workflow for Health Risk Screening. As seen in FIG. 12, steps in this workflow include:
Enter Screening Area
If there are no symptoms, person can proceed
If there are symptoms, person remains in the screening area and scanned
Person is monitored for the following triggers:
   Elevated Temperature
   Listen for Cough, Sneeze, Sniffling
   Listening for shortness of breath
If two of the five triggers are detected, person may go to secondary screening point and have their temperature manually taken.

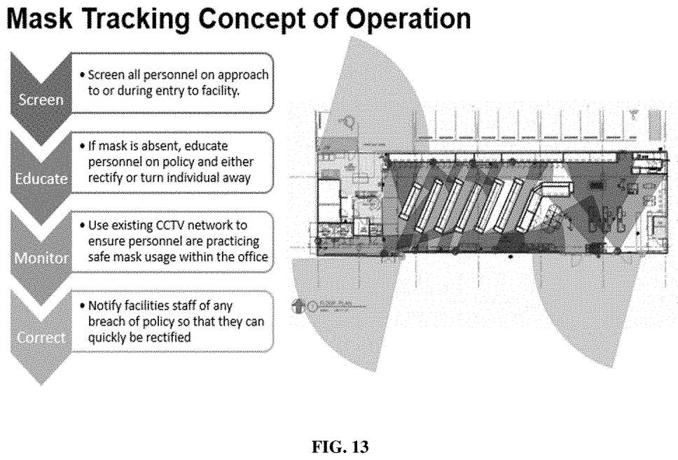

FIG. 13 is a diagram illustrating a workflow for Mask Tracking. As seen in FIG. 13, the steps in this workflow include:
Screen: Screen all personnel on approach to or during entry to facility.
Educate: If mask is absent, educate personnel on policy and either rectify or turn individual away
Monitor: Use existing CCTV network to ensure personnel are practicing safe mask usage within the site
Correct: Notify facilities staff of any breach of policy so that they can quickly be rectified

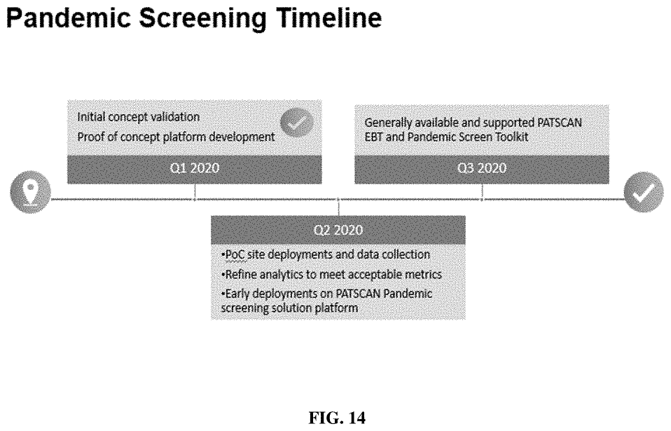

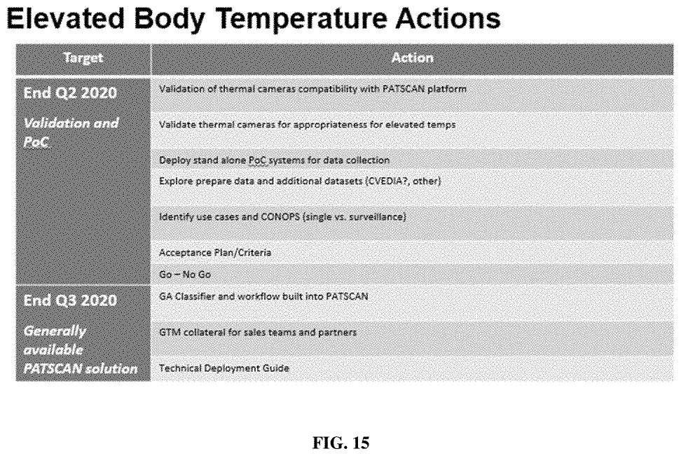

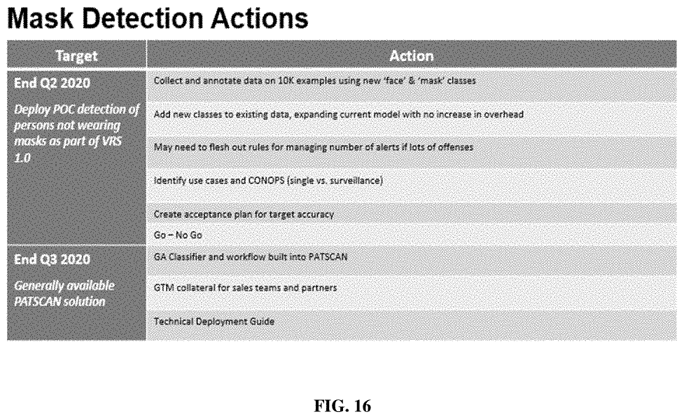

The modules for potential health risk screening as shown in FIG. 11 is also useful for pandemic screening. FIG. 14 is a diagram illustrating potential deadlines for implementing Pandemic Screening modules. FIG. 15 is a diagram illustrating actions related to Elevated Body Temperature tasks. FIG. 16 is a diagram illustrating actions related to Mask Detection tasks.

### Modules for Violence Detection:

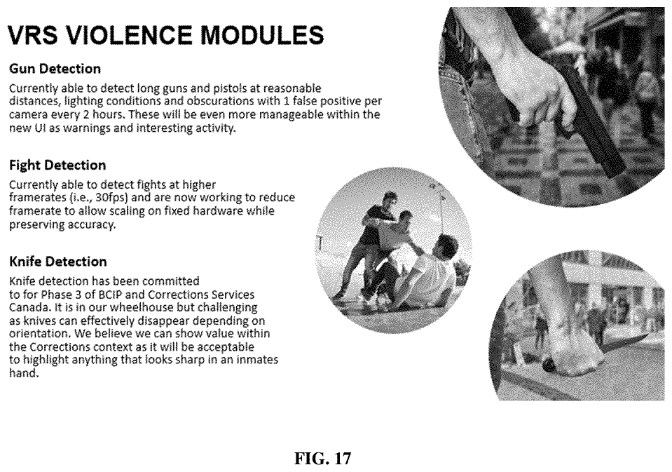

FIG. 17 is a diagram illustrating modules for Violence Detection. As seen in FIG. 17, these modules support:
Gun Detection: Ability to detect long guns and pistols at reasonable distances, lighting conditions and obscurations with 1 false positive per camera every 2 hours as an example.
Fight Detection: Ability to detect fights at higher framerates (i.e., 30 fps) as well as on lower framerates.
Knife Detection: Ability to highlight sharp objects on subjects, which is valuable in a Corrections context.

### Fight Detection Module:

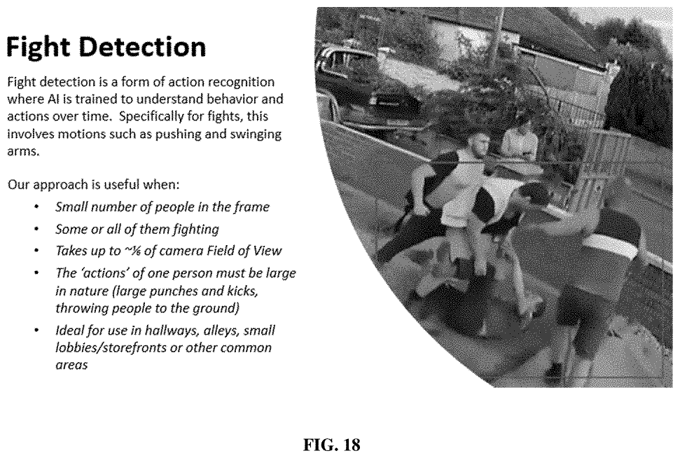

Fight detection is a form of action recognition where AI is trained to understand behavior and actions over time. Specifically for fights, this involves motions such as pushing and swinging arms. FIG. 18 is a diagram illustrating a Fight Detection module. This proposed approach is most useful when:
There are a few people in the frame.
Some or all of them fighting.
Takes up to ˜⅙ of camera Field of View
The ‘actions’ of one person must be large in nature (large punches and kicks, throwing people to the ground)
Ideal for use in hallways, alleys, small lobbies/storefronts or other common areas

### Disturbance Detection Module:

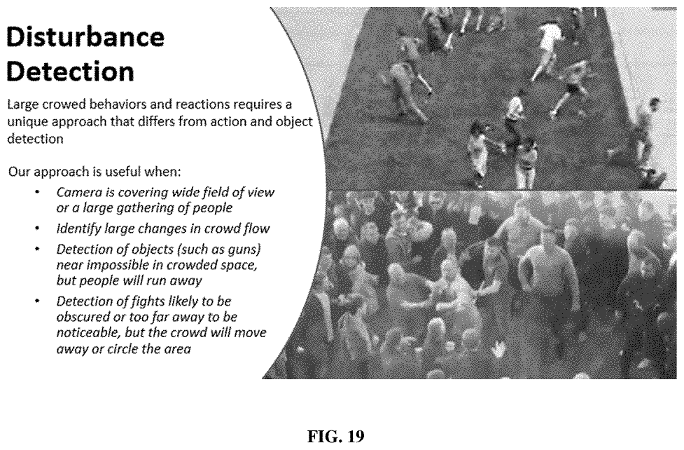

Large crowd behaviors and reactions may require a unique approach that differs from action and object detection. FIG. 19 is a diagram illustrating a Disturbance Detection module. This proposed approach is useful when:
Camera is covering wide field of view or a large gathering of people
Identify large changes in crowd flow
Detection of objects (such as guns) near impossible in crowded space, but people will run away, as a secondary indication of possible a possible firearm.
Detection of fights likely to be obscured or too far away to be noticeable, but the crowd will move away or circle the area

### VRS (Video Recognition System) Facilities Support:

Knowledge of how employees, patrons and even the public use and interact with the space around them is fundamental to answer such key questions as:
What should we clean?
What parts of our facility do we need to heat and cool?
How do we effectively secure our facility?

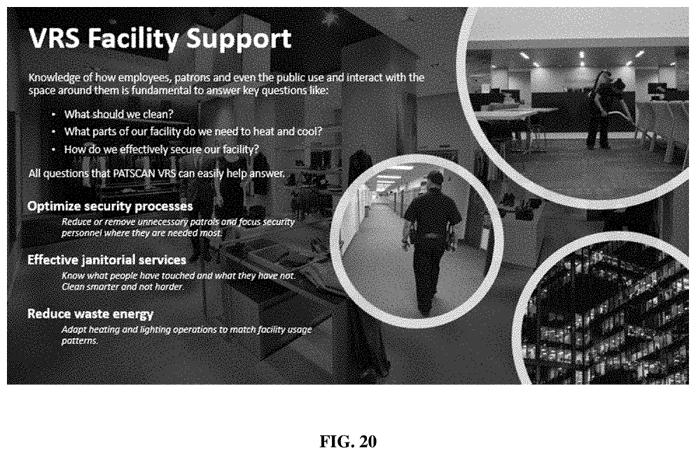

FIG. 20 is a diagram illustrating modules for Facility Support. The system can provide facilities support and address the following:
Optimize security processes by reducing or removing unnecessary patrols and focusing security personnel where they are needed most.
Make janitorial services more effective through knowing what people have touched and what they have not.
   Reduce waste energy by adapting heating and lighting operations to match facility usage patterns.

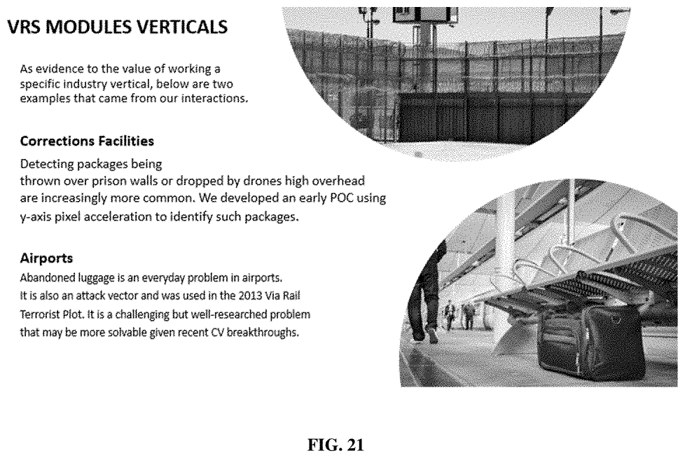

FIG. 21 is a diagram illustrating modules to support additional verticals. As seen in FIG. 21, the system can support interactions to industry specific verticals such as correction facilities and airports:

Corrections Facilities
Detection of packages being thrown over prison walls or dropped by drones high overhead. An embodiment may use y-axis pixel acceleration detection to identify such packages.

Airports
Abandoned luggage is an everyday problem in airports. It is also an attack vector and was used in the 2013 Via Rail Terrorist Plot. An embodiment may use Computer Vision with AI to detect these

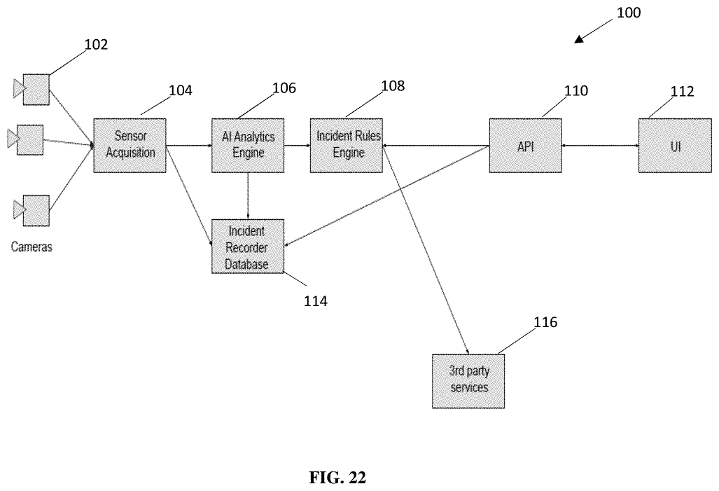

FIG. 22 is a system diagram of an exemplary threat detection system. As seen in FIG. 22, threat detection system 100 consist of one or more cameras 102 configured to record video data (images and audio). Cameras 102 is connected to sensor or sensor acquisition module 104. Once the data is acquired, the data is sent simultaneously to an AI Analytics Engine 106 and Incident Recorder Database 114. AI Analytics Engine 106 analyzes the data with input from an Incident Rules Engine 108. Thereafter, the data is sent to an application program interface (API) 110 or sent to 3rd party services 116. The output form the API 110 will be sent to a user interface (UI) 112 or graphical user interface (GUI). Furthermore, the output from the API 110 and AI Analytics Engine 106 will be further recorded at the Incident Recorder Database 114.

In further embodiments, disclosed herein is a multi-sensor threat detection system used for detection of concealed and visible threats. The system comprises a processor to compute and process data from sensors in an environment, an imaging system configured to capture image data and a graphical user interface (GUI) to provide an update of real-time data feeds based on the processed feeds.

The imaging system of the multi-sensor threat detection system is an optical camera, thermal camera, sensor camera or a sensor module. The system further comprises a smoke or fire sensor, a fight detection module, an elevated body temperature sensing module.

The multi-sensor threat detection system further comprises a health risk screening module, the health risk screening module configured to test body temperature and listen for coughing, sneezing, sniffling and shortness of breath and report these conditions to the graphical user interface (GUI). The system further comprises a mask detection module, the mask detection module configured to detect the presence or absence of a mask on a subject in view of at least one optical camera and report results to the graphical user interface (GUI). The system further comprises a social distancing detection module, the social distancing module configured to detect the distance between subjects in view of at least one optical camera, determine whether this distance falls below appropriate social distancing rules and report these results to the graphical user interface (GUI).

In further embodiments, disclosed herein is a computer-implemented method for reporting real-time threat, using a multi-sensor threat detection system, the method comprising receiving image data from an imaging system of the multi-sensor threat detection system, processing the data using the processor and at least one artificial intelligence algorithm, displaying the data on a graphical user interface (GUI) and sending an alert warning when a threat is identified. The alert warning is sent to security personnel, the command center and users of the threat detection system.

The functions described herein may be stored as one or more instructions on a processor-readable or computer-readable medium. The term “computer-readable medium” refers to any available medium that can be accessed by a computer or processor. By way of example, and not limitation, such a medium may comprise RAM, ROM, EEPROM, flash memory, CD-ROM or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor. A “module” can be considered as a processor executing computer-readable code.

A processor as described herein can be a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, or microcontroller, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, any of the signal processing algorithms described herein may be implemented in analog circuitry. In some embodiments, a processor can be a graphics processing unit (GPU). The parallel processing capabilities of GPUs can reduce the amount of time for training and using neural networks (and other machine learning models) compared to central processing units (CPUs). In some embodiments, a processor can be an ASIC including dedicated machine learning circuitry custom-build for one or both of model training and model inference.

The disclosed or illustrated tasks can be distributed across multiple processors or computing devices of a computer system, including computing devices that are geographically distributed.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

While the foregoing written description of the system enables one of ordinary skill to make and use what is considered presently to be the best mode thereof, those of ordinary skill will understand and appreciate the existence of variations, combinations, and equivalents of the specific embodiment, method, and examples herein. The system should therefore not be limited by the above described embodiment, method, and examples, but by all embodiments and methods within the scope and spirit of the system. Thus, the present disclosure is not intended to be limited to the implementations shown herein but is to be accorded the widest scope consistent with the principles and novel features disclosed herein.

What is claimed is:

1. A multi-sensor threat detection system used for detection of concealed and visible threats, the system comprising:
a processor to compute and process data from sensors in an environment;
an imaging system configured to capture image data; and
a graphical user interface (GUI) configured to provide an update of real-time data feeds based on the processed data.

2. The system of claim 1 wherein the imaging system is an optical camera.

3. The system of claim 1 wherein the imaging system is a thermal camera.

4. The system of claim 1 wherein the imaging system is a sensor camera or sensor module.

5. The system of claim 1 further comprising a smoke or fire sensor.

6. The system of claim 1 further comprising a fight detection module.

7. The system of claim 1 further comprising a disturbance detection module.

8. The system of claim 1 further comprising an elevated body temperature sensing module.

9. The system of claim 1 further comprising a health risk screening module, the health risk screening module configured to test body temperature and listen for at least one of coughing, sneezing, sniffling and shortness of breath and report these conditions to the graphical user interface (GUI).

10. The system of claim 1 further comprising a mask detection module, the mask detection module configured to detect the presence or absence of a mask on a subject in view of at least one optical camera and report results to the graphical user interface (GUI).

11. The system of claim 1 further comprising a social distancing detection module, the social distancing module configured to detect the distance between subjects in view of at least one optical camera, determine whether this distance falls below distancing rules and report these results to the graphical user interface (GUI).

12. A computer-implemented method for reporting real-time threats, using a multi-sensor threat detection system, the method comprising:
receiving image data from an imaging system of the multi-sensor threat detection system;
processing the data using the processor and at least one artificial intelligence algorithm;
displaying the data on a graphical user interface (GUI); and
sending an alert warning when a threat is identified.

13. The system of claim 12 wherein the alert warning is sent to security personnel, the command center and users of the threat detection system.
